(19) United States
(12) Patent Application Publication
(10) Pub. No.: US 2011/0318438 A1
(43) Pub. Date: Dec. 29, 2011

(54) PHARMACOLOGICALLY EFFECTIVE COMPOSITION OF HERBS FOR TREATMENT ON SYMPTOMS OF INFLUENZA AND COLDS

(76) Inventor: WILLIAM KAM KIT CHUNG, (US)

(21) Appl. No.: 12/822,225

(22) Filed: Jun. 24, 2010

(51) Int. Cl. A61K 36/00; A61P 31/16

(52) U.S. Cl. 424/777

(57) ABSTRACT

A pharmacologically effective composition of herbs is provided for symptom relieving for influenza and colds. The composition that is antiviral, antibacterial and does not contain any undesirable stimulants such as caffeine.

### CROSS-REFERENCE TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

The invention relates to use a pharmacologically effective herbal composition for treatment of colds and Influenza.

Colds and influenza are major causes of illness and loss of productivity throughout the United States and the rest of the world. A significant portion of colds are thought to be caused by viruses and result in other serious illness. It has long been considered desirable to provide an effective treatment for a wide variety of illnesses caused by viruses and bacteria, that are potent antiviral and antimicrobial agents, but also can relieve symptoms. Some Chinese medicine herbal formulas can be at least somewhat effective, but they typically contain caffeine or other components undesirable to a large segment of the population. It is desirable to provide effective herbal compositions without caffeine or other stimulants.

Under the US Patent Classifications, the invention may classified under 424/725 Plant material or plant extract of undetermined constitution as active ingredient, 424/765 Drug, bio-affecting and body treating compositions containing or obtained from Roseaceae, 424/773 Drug, bio-affecting and body treating compositions containing or contained from a root, bulb, tuber, corm, or rhizome, 424/777 Drug, bio-affecting and body treating compositions containing or obtained from a fruit, 424/778 Drug, bio-affecting and body treating compositions containing or obtained from a flower or blossom.

### BRIEF SUMMARY OF THE INVENTION

According to the present invention a pharmacologically effective composition which is a broad spectrum antiviral and antimicrobial is provided which is herbal in nature and does not contain caffeine or like undesirable components. Fructus Forsythiae, Scutellaria Baicalensis Georgi, Eriobotryae Folium, Platycodon Grandiflorum, Trichosanthis Pericarpium are the major ingredients, comprising collectively a majority of the composition. The composition may be used in the treatment of colds and influenza. The compositions according to the present invention are antiviral, antibacterial, and symptom relieving.

According to one aspect of the present invention a pharmacologically effective composition is provided comprising as the active ingredients: a mixture of Fructus Forsythiae, Scutellaria Baicalensis Georgi, Eriobotryae Folium, Platycodon Grandiflorum, Trichosanthis Pericarpium, Radiux Bupleuri and Flos Lonicerae, in a pharmacologically effective amount. The components of the mixture preferably have the following contributions expressed in approximate weight percent:

### Fructus Forsythiae 350 g,

### Scutellaria Baicalensis Georgi 350 g,

### Eriobotryae Folium 350 g,

### Platycodon Grandiflorum 350 g,

### Trichosanthis Pericarpium 350 g,

### Radiux Bupleuri 200 g,

### Flos Lonicerae 200 g.

Preferably the composition active ingredients consist essentially of the herbs set forth above.

The invention also relates to a method of substantially eliminating or ameliorating viral and microbial illness of human patients comprising the step of administering to a human patient in need of treatment an effective amount of a pharmacological composition such as set forth above. The invention also relates to a method of substantially eliminating or ameliorating in a human patient colds, influenza and cough, comprising the step of administering to a human patient in need of treatment an effective amount of a pharmacological composition as set forth above.

The invention also relates to a method of substantially eliminating or ameliorating viral and microbial illness of human patients comprising the step of administering to a human patient a pharmacologically effective composition comprising active ingredients consisting essentially of Fructus Forsythiae, Scutellaria Baicalensis Georgi, Eriobotryae Folium, Platycodon Grandiflorum, Trichosanthis Pericarpium making up the majority of the active ingredients, along with other anti-microbial herbal agents, and herbal agents for relief of aches and pains, sore throat, and to reduce fever.

### BRIEF DESCRIPTION OF THE SEVERAL VIEWS OF THE DRAWING

Not Applicable.

### DETAILED DESCRIPTION OF THE INVENTION

This invention relates to a herbal composition for treatment of colds and influenza. The compositions according to the present invention use Fructus Forsythiae, Scutellaria Baicalensis Georgi, Eriobotryae Folium, Platycodon Grandiflorum, Trichosanthis Pericarpium, Radiux Bupleuri and Flos Lonicerae as the major ingredients. These herbs have been used individually or combination in traditional Chinese medicine for colds, virus, and infection. These components are utilized in the composition according to the present invention along with other potent herbal antimicrobial agents, and along with herbs for relief of aches and pains, and sore throat, and to reduce fevers. The herbal compositions according to the present invention are antiviral, antibacterial, and symptom relieving on colds and influenza.

According to the present invention a pharmacologically effective composition is provided comprising as active ingredients a mixture of Fructus Forsythiae, Scutellaria Baicalensis Georgi, Eriobotryae Folium, Platycodon Grandiflorum, Trichosanthis Pericarpium, Radiux Bupleuri and Flos Lonicerae in a pharmacologically effective amount. The components of the mixture preferably have the following contributions expressed in approximate weight percent: Fructus Forsythiae 16.3%, Scutellaria Baicalensis Georgi 16.3%, Eriobotryae Folium 16.3%, Platycodon Grandiflorum 16.3%, Trichosanthis Pericarpium 16.3%, Radiux Bupleuri 9.25% and Flos Lonicerae 9.25%.

“Fructus Forsythiae” is also known as ‘Lian Qiao’. The fruit of the forsythia is used medicinally. Green forsythia fruit is preferred over yellow forsythia fruit. The fruit is steamed, dried in the sun, and the seeds removed from the fruit before used in herbal remedies. In traditional Chinese medicine, forsythia is considered to be bitter and cold, and is associated with the Heart, Lung and Gall Bladder meridians. It clears heat and expels wind. Forsythia is typically used to detoxify the body, and to treat fevers, headaches and excessive thirst.

“Scutellaria Baicalensis Georgi” is also known as ‘Huang Qi’ or ‘Astragalas’. Scutellaria Baicalensis Georgi contains numerous compounds, including flavonoids, polysaccharides, amino acids and trace minerals. Traditionally, it was used in China for conditions such as night sweats, diarrhea, and qi deficiency. Preliminary studies conducted in Asia have determined that Scutellaria Baicalensis Georgi can enhance immune function and lengthen the lives of people with cancer. Other studies suggest that it may prevent some infections in people undergoing dialysis for kidney failure.

“Eriobotryae Folium Japonicae” is also known as ‘Pi Pa Ye or Chinese Loquat Leaf’. Eriobotryae Folium has been used as a medicinal plant for a long time, and it is known to have many physiological actions such as anti-inflammatory, antitussive, expectorant and anti-diabetic. Eriobotryae Folium transforms Phlegm, Calms cough and Harmonizes the Stomach.

“Platycodon Grandiflorum” is also known as ‘Jie Geng’. Platycodon is a perennial plant that the main and secondary roots are used medicinally. The healing effects of platycodon are derived from its volatile oil and other compounds, including saponins and plant sterols. Traditionally, platycodon has been used as an expectorant and to treat bronchitis, tonsillitis, sore throat and other respiratory conditions.

“Trichosanthis Pericarpium” is also known as ‘Gua Lou Pi’. Different parts of trichosanthes are used for different ailments. In traditional Chinese medicine concepts, trichosanthes seeds (gua lou ren) are used to treat dry stools and mild forms of constipation, and to help wounds heal. Trichosanthes root (tian hua fen) treats lung heat conditions that have phlegm and dryness, and reduces toxins manifested by sores and inflammation, especially breast abscesses. Both the seeds and root can be used externally or internally as needed. Tricosanthes peel treats the stomach and aids in the circulation of qi. Many Chinese herbalists also use tricosanthes for high blood pressure and high blood lipid levels.

“Radiux Bupleuri” is also known as ‘Chai Hu’. Bupleurum is an upright-growing plant native to China, Japan and central Europe, but now widely dispersed throughout the U.S. The root is used medicinally. The active ingredients in bupleurum root include saponins and plant sterols, which have been shown to lower fevers and reduce inflammation in animal studies. It is used for a variety of conditions, including inflammatory conditions, angina, nausea, vomiting and fever. It also strengthens the stomach and intestines and promotes blood circulation to the liver.

“Flos Lonicerae” is also known as ‘Jin Yin Hua’. Different parts of the Flos Lonicerae are used to treat different conditions. The flower buds are gathered in the beginning of summer, then dried in the shade before being used in herbal remedies. They can be used raw, or after being fried or distilled. In traditional Chinese medicine, Flos Lonicerae has sweet and cold properties, and is associated with the Lung, Stomach and Large Intestine meridians. It is used to clear heat and remove toxins. Generally, Flos Lonicerae has been employed to treat a variety of conditions, ranging from fevers, ulcers, inflammation and sore throats to skin infections. It is also used (in conjunction with coptis and pulsatilla) to treat diarrhea caused by toxic heat. Flos Lonicerae may be applied internally or externally.

In addition to the active ingredients, the herbal complexes utilized according to the present invention may have any number of substantially inert ingredients which will vary depending upon the particular form by which the complex will be administered. Normally the complex is administered in the form of ingestible tablets or capsules which are swallowed with water, although the complex active ingredients may be mixed with food or beverage items and eaten or drunk, or in extreme cases may be introduced directly into the bloodstream using a hypodermic needle, I.V., or the like. The dose may vary depending upon the size, age, and condition of the patient being treated and the particular percentages of components herbal complex utilized.

### PHARMACEUTICAL METHODS

Step 1: Take a pharmacologically effective composition of Fructus Forsythiae 350 g, Scutellaria Baicalensis Georgi 350 g, Eriobotryae Folium 350 g, Platycodon Grandiflorum 350 g, Trichosanthis Pericarpium 350 g, Radiux Bupleuri 200 g, Flos Lonicerae 200 g and add 100,000 ml water to boiling for one hour and then are filtrated. Then add another 100,000 ml water to the above composition again and bring to the boiling temperature for another one hour and filtrated. The aqueous solution from both process are mixed together and filtered and the filtrate was concentrated until a density of 1.30 -1.34 is reached.
Step 2: After the concentrated solution above step 1 is cooled, ethyl alcohol is added until the amount of ethyl alcohol is 70-80%. The mixture is stir well and then allowed to sit at room temperature for twenty four (24) hours followed by filtration to remove all solid matter from the mixture. Recycle the ethyl alcohol from the liquid, and the obtained filtrate is continuously concentrated into a solution with a density of 1.34-1.37.
Step 3: The concentrated solution from Step 2 is filtrated for impurities, then dried in a vacuum oven at 70° C. temperature into powder form, then this pharmacologically effective composition could be made into any regular medicinal types such as tablets, capsules or pellets and packed.

What is claimed is:

1. A pharmacologically effective composition comprising as active ingredients a mixture of Fructus Forsythiae, Scutellaria Baicalensis Georgi, Eriobotryae Folium, Platycodon Grandiflorum, Trichosanthis Pericarpium, Radiux Bupleuri and Flos Lonicerae in a pharmacologically effective amount, wherein the components of the mixture have the following contributions expressed in approximate weight percent.

2. A composition as recited in claim 1 wherein said active ingredients consist essentially of Fructus Forsythiae, Scutellaria Baicalensis Georgi, Eriobotryae Folium, Platycodon Grandiflorum, Trichosanthis Pericarpium, Radiux Bupleuri and Flos Lonicerae.

3. A composition as recited in claim 2 wherein the components of the mixture have the following contributions expressed in approximate weight percent.

4. A method of substantially eliminating or ameliorating viral and microbial illness of human patients comprising the step of administering to a human patient in need of treatment an effective amount of the pharmacological composition of claim 1.

5. A method of substantially eliminating or ameliorating viral and microbial illness of human patients comprising the step of administering to a human patient in need of treatment an effective amount of the pharmacological composition comprising as active ingredients a mixture of Fructus Forsythiae, Scutellaria Baicalensis Georgi, Eriobotryae Folium, Platycodon Grandiflorum, Trichosanthis Pericarpium, Radiux Bupleuri and Flos Lonicerae in a pharmacologically effective amount.

6. A method of substantially eliminating or ameliorating viral and microbial illness of human patients comprising the step of administering to a human patient in need of treatment an effective amount of the pharmacological composition of claim 2.

7. A method of substantially eliminating or ameliorating viral and microbial illness of human patients comprising the step of administering to a human patient in need of treatment an effective amount of the pharmacological composition of claim 3.

8. A method of substantially eliminating or ameliorating in a human patient colds and influenza comprising the step of administering to a human patient in need of treatment an effective amount of the pharmacological composition of claim 1.

9. A method of substantially eliminating or ameliorating in a human patient colds and influenza comprising the step of administering to a human patient in need of treatment an effective amount of the pharmacological composition comprising as active ingredients a mixture of Fructus Forsythiae, Scutellaria Baicalensis Georgi, Eriobotryae Folium, Platycodon Grandiflorum, Trichosanthis Pericarpium, Radiux Bupleuri and Flos Lonicerae in a pharmacologically effective amount.

10. A method of substantially eliminating or ameliorating in a human patient colds and influenza comprising the step of administering to a human patient in need of treatment an effective amount of the pharmacological composition of claim 2.

11. A method of substantially eliminating or ameliorating in a human patient colds and influenza comprising the step of administering to a human patient in need of treatment an effective amount of the pharmacological composition of claim 3.

12. A composition as recited in claim 1 wherein the components of the mixture have the following contributions expressed in weight percent.

13. A composition as recited in claim 2 wherein the components of the mixture have the following contributions expressed in weight percent.

14. A method of substantially eliminating or ameliorating viral and microbial illness of human patients comprising the step of administering to a human patient a pharmacologically effective composition having active ingredients consisting essentially of Fructus Forsythiae, Scutellaria Baicalensis Georgi, Eriobotryae Folium, Platycodon Grandiflorum, Trichosanthis Pericarpium, Radiux Bupleuri and Flos Lonicerae making up the majority of the active ingredients, along with other anti-microbial herbal agents, and herbal agents for relief of aches and pains, sore throat, and to reduce fever.

15. A method as recited in claim 14 wherein said method is practiced by administering a pharmacologically effective composition in which the Fructus Forsythiae, Scutellaria Baicalensis Georgi, Eriobotryae Folium, Platycodon Grandiflorum, Trichosanthis Pericarpium, collectively comprise between 80% of the active ingredients.

16. A method of substantially eliminating or ameliorating viral and microbial illness of human patients comprising the step of administering to a human patient a pharmacologically effective composition comprising active ingredients comprising Fructus Forsythiae, Scutellaria Baicalensis Georgi, Eriobotryae Folium, Platycodon Grandiflorum, Trichosanthis Pericarpium making up the majority of the active ingredients, along with other anti-microbial herbal agents, and herbal agents for relief of aches and pains, sore throat, and to reduce fever; and wherein said composition is devoid of caffeine or like stimulants.
